(19) United States
(12) Patent Application Publication
(10) Pub. No.: US 2005/0090457 A1
(43) Pub. Date: Apr. 28, 2005

(54) Method of treating mammals with genistein and/or genistein analogues

(75) Inventors: Inez Schoenmakers, Utrecht (NL); Zhi Chao Dang, Leiden (NL); Clemens Waitherus Gerardus Maria Lowik, Oegstgeest (NL); Adrianus Lambertus Bertholdus Van Helvoort, Wageningen (NL); Robert Johan Joseph Hageman, Wageningen (NL); Marijan Hest Gros-Van, Renkum (NL)

Correspondence Address: YOUNG & THOMPSON, 745 SOUTH 23RD STREET, 2ND FLOOR, ARLINGTON, VA, 22202, US

(73) Assignee: N.V. NUTRICIA, ZOETERMEER (NL)

(21) Appl. No.: 10/504,713

(22) PCT Filed: Feb. 14, 2003
(86) PCT No.: PCT/NL03/00109

(30) Foreign Application Priority Data
Feb. 15, 2002 (EP) 02075611.0

(51) Int. Cl. A61K 31/7048; A61K 31/353

(52) U.S. Cl. 514/27; 514/456

(57) ABSTRACT

The present invention is concerned with a method of controlling the plasma genistein concentration in mammals in order to avoid activation of the peroxisome proliferator-activated receptor γ (PPARγ), said method comprising the steps of: a. assessing the genistein blood serum concentration of the mammal; b. if needed, administering to said mammal a genistein component in an amount sufficient to maintain the genistein blood serum concentration at a level between 0.02 and 3 μM during at least 8 hours, preferably at least 16 hours of each day; c. repeating steps a. and b. during a period of at least 30 days with intervals of no more than 3 days. The present method is particularly suited for preventing obesity and diseases or conditions in which bone tissue is lost.

### TECHNICAL FIELD OF THE INVENTION

The present invention relates to a method of in vivo administration of defined amounts of genistein in order to maintain the plasma genistein concentration within defined ranges in mammals. More particularly, the present invention is concerned with a method of controlling the plasma genistein concentration in order to stimulate osteogenesis whilst at the same time preventing adipogenesis by avoiding activation of the peroxisome proliferator-activated receptor γ (PPARγ).

The present method is particularly suited for preventing obesity and diseases or conditions in which bone tissue is lost (such as in osteoporosis, Paget's disease, osteolytic metastasis in cancer patients, osteodystrophy in liver disease, altered bone metabolism caused by renal failure or haemodialysis, bone fracture, bone surgery, pregnancy, lactation, anorexia nervosa, or immobile, non-weight bearing, malnourished or weight losing persons).

### BACKGROUND OF THE INVENTION

Isoflavones have been advocated for the prevention of a variety of diseases. The actual functionality of these isoflavones is however poorly understood. They are known to bind to the two known estrogen receptors (ER's) ERα and ERβ, mediating estrogenic effects (Anderson, 2001). In addition, genistein is also an inhibitor of protein tyrosin kinase. Isoflavones have however also other anti-estrogenic and non-ER dependent effects. The mechanisms of these effects are still unknown (Anderson, 2001; Aldercreutz and Mazur, 1998; Kim et al., 1998).

The optimum dosage for the prevention of bone loss and obesity has not been determined, since dose-effect relationships have not been well established in vivo or in vitro. This is believed to be due to the fact that many known in vitro systems have a low reproducibility and/or sensitivity.

Anderson et al., Proc. Soc. Exp. Biol. Med. 1998: 217(3), 345-350, describe a positive effect of genistein on the retention of bone mass in ovariectomised, lactating rats at dosages of 6.2-19.7 μmole genistein per kg bodyweight per day. A lower response is reported for higher dosages (61.7 μmole genistein per kg bodyweight per day) in addition to a negative impact on uterine weight. The genistein dosages employed in this study are extremely high and exceed the daily amounts of genistein, calculated on bodyweight, that are normally consumed by humans.

Nuttall et al., “Is there a therapeutic opportunity to either prevent or treat osteopenic disorders by inhibiting marrow adipogenesis”, Bone 27(2), (2000), 177-184, present a review that explores the stromal cell's differentiation plasticity in the context of osteoporosis and other metabolic bone disorders. A reciprocal relationship is postulated to exist between the adipocyte and osteoblast phenotypes. The signal transduction pathways implicated in this process are evaluated as potential targets for therapeutic intervention and drug design. It is observed in the article that in vitro and in vivo studies demonstrate that ligands binding to the PPARs, glucocorticoid, estrogen, androgen and vitamin D₃ receptors regulate bone marrow stromal cell adipogenesis and osteogenesis. In addition it is observed that drugs belonging to the thiazolidinediones promote bone marrow stromal cell adipogenesis at the expense of osteogenesis.

The family of transcription factors known as the peroxisome proliferator-activated receptors (PPARs) plays a central role in regulating the storage and catabolism of dietary fats. The PPARs were cloned less than a decade ago as orphan members of the nuclear receptor gene family that includes the receptors for the steroid, retinoid and thyroid hormones. It has been suggested to use PPARs as molecular targets for the development of drugs to treat human metabolic diseases.

There are 3 PPAR subtypes, which are the products of distinct genes and are commonly designated PPARα, PPARγ and PPARδ. In common with other members of the nuclear receptor gene family, the PPARs are ligand-activated transcription factors. The binding of agonist ligands to the receptor results in changes in the expression level of mRNAs encoded by PPAR target genes. This process is known as “transactivation”, and cell-based assays have been developed which can be used to monitor this functional activity.

The biology of the PPARs has been driven, in large part, by the availability of potential and selective ligands for the receptors. Through the use of binding and functional assays, several groups have reported the identification and optimization of PPAR ligands for each of the 3 subtypes. These chemical tools have been used in a “reverse endocrinology” approach to uncover the role of the PPARs in human physiology and disease processes.

PPARγ is the most extensively studied of the 3 PPAR subtypes to date. PPARγ is a critical transcription factor in the regulation of adipocyte differentiation. It has been found that forced expression of PPARγ in fibroblasts in the presence of weak PPARγ activators resulted in differentiation of the cells to adipocytes. Data available to date show that PPARγ plays a pivotal role in the adipogenic signaling cascade and also suggest that the receptor can influence the production and cellular uptake of its own activators.

Known natural PPARγ ligands include thiazolidines, a number of polyunsaturated fatty acids, eicosanoid derivatives and J-series of prostaglandins derived from PGD₂, that binds and activates the receptor at micromolar concentrations.

Although the stimulation PPARγ is associated with decreased osteogenesis and increased adipogenesis, the activation of PPARγ also leads to health benefits. The most extensively studied therapeutic utility for PPARγ agonists has been in the treatment of type 2 diabetes. In addition studies have been conducted that show that PPARγ agonists decrease plasma levels of triglycerides, cholesterol, and free fatty acids in various animal models of dyslipidemia. Furthermore there are indications that PPARγ agonists may have antihypertensive, anti-inflammatory and anti-tumor effects.

Liang et al, “Suppression of inducible cyclooxygenase and nitric oxide synthase through activation of PPARγ by flavonoids in mouse macrophages”, FEBS Letters 496 (2001) 12-18, report that the flavonoids apigenin, chrysin and kaempferol may act as allosteric effectors that are able to bind and activate PPARγ. Genistein did in this study however not stimulate PPARγ activity at a concentration of 10 μM. PPARγ binding studies for genistein were not performed. It is suggested that these flavonoids might have therapeutic applications in inflammatory diseases, such as atherosclerosis and rheumatoid arthritis.

U.S. Pat. No. 5,506,211 (The UAB Research Foundation) describes a method for use in inhibiting osteoclast activity which leads to bone degradation, comprising contacting an osteoclast with a composition comprising an amount of a genistein-glucoside conjugate that occurs naturally in soy. The advocated daily doses of genistein-glucoside conjugate are in the range of 2-50 mg.

U.S. Pat. No. 5,953,996 (Taishi Food Company) describes a method for accelerating ossification and preventing reduction of bone salt content which comprises administering to a mammal in need thereof an effective amount of a composition containing synergistic effective amounts of genistein and a zinc salt. It is observed that the inventors have found that genistein and zinc may enhance osteogenesis. The compositions described are preferably ingested so that the concentration of genistein in blood in one hour after ingestion may be 10⁻⁷ M or more.

WO 99/66913 (Sigma-Tau Healthscience) describes a composition, which comprises as active ingredients propionyl L-carnitine and genistein for the prevention and/or therapeutic treatment of osteoporosis and menopause syndrome.

WO 01 74345 A (Ingram Jonathan) describes a method of suppressing weight gain, inducing weight loss, or imparting a feeling of gastric fullness, comprising administering at least one isoflavone, such as genistein. The isoflavone may be administered in a range of 5-500 mg/day.

U.S. Pat. No. 6,326,366 (Protein Technologies International) is concerned with a hormone replacement theray regimen comprising co-administering a therapeutically effective amount of a combination of mammalian estrogen and isoflavone (e.g. genistein). The advocated daily dosage of isoflavone is in the range of 20-1000 mg.

EP-A 0 829 261 relates to a composition for fat-degradation in a fat cell which comprises at least one isoflavone and/or a derivative thereof. The isoflavone is suitably selected from the group consisting of daidzein, daidzin, genistein and genistin. In the patent application, a dosage dependent stimulation of lipolysis in adipocytes is observed after exposure to 3 to 100 micro M genistein. No effects were observed at 0.3 and 1 μM.

Harmon et al. (Am J Physiol Cell Physiol. 2001 April;280(4):C807-13) report the results of a study into the differential effects of flavonoids on 3T3-L1 adipogenesis and lipolysis. It is concluded that the flavonoids genistein and naringenin inhibit proliferation of preadipocytes in a time- and dose-dependent manner. When added to 2-day postconfluent preadipocytes at the induction of differentiation, genistein was found to inhibit PPAR-γ expression. On page C812 it is observed “Given that genistein promotes lipolysis and inhibits adipogenesis in cell culture, we anticipate that genistein will act similarly in vivo and potentially promote loss of body fat”. It is furthermore stated: “a non-toxic pharmacological dose of genistein, 8 mg/kg body weight, elevates serum genistein to the 10 μM range . . . , a level sufficient to inhibit adipogenesis in 3T3-L1 cells.

Dang et al. (J. of Bone and Mineral Res., 2000 September;15(1), S496) describe the results of a study into the effects of genistein and 17β-estradiol on osteoblastogenesis and adipogenesis. Two of the present inventors are mentioned as authors of this publication. The abstract presents data on the effects of exposure of KS485 cells to low concentrations of genistein. Although this experimental work provided a foundation for the present invention, it was not known at the time that higher concentrations of genistein produce an adverse and opposite effect to those observed at lower concentrations. Wang et al. (J. Biol. Chem. 1999 Nov. 5;274(45):32159-66) report the results of a study that show the ability of a tyrosine kinase inhibitor to block adipogenesis in response to dexamethasone and methylisobutylxanthine and provide compelling evidence for a central role of the tyrosine kinase Syk in the G_{S}α-mediated regulation of adipogenesis. In the article the following observations are made: “The dose dependence with respect to the effects of genistein on D/M induced adipogenesis was explored. At 100 μM genistein effectively abolishes adipogenic conversion. Both 10 and 33 μM concentrations of the tyrosine kinase inhibitor provoke a marked inhibition of adipogenesis. at 3.3 μM, the lowest concentration tested, genistein displays only a weak inhibitory influence on the differentiation of the 3T3-L1 cels in response to the inducers D/M. No references can be found in any of the aforementioned prior art documents as to the ability of isoflavones, and particularly of genistein, to bind and activate PPARγ, i.e. to act as a PPARγ agonist. Although the use of genistein components has been advocated for the treatment of osteoporosis and a wide variety of other disorders and diseases, the actual functionality of these isoflavones is poorly understood. The dose-effect relationships for genistein and osteoporosis are not well established and, as will be shown below, are intrinsically flawed because they have not taken account of the PPARγ mediated effect of genistein on adipogenisis.

### SUMMARY OF THE INVENTION

The inventors have discovered that genistein components may suitably be used to treat or prevent a variety of diseases including osteoporosis, obesitas and/or syndrome X (insulin resistance syndrome), provided these isoflavones are administered in accordance with a regime that does not lead to plasma genistein levels that are sufficiently high to activate the PPARγ receptor. Despite the large number of prior art publications that deal with the favourable effect of genistein on e.g. osteogenesis and/or adipogenesis, it has not been recognized before that it is crucial to avoid that plasma genistein levels reach a level that is sufficient to activate the PPARγ receptor in order to prevent inhibition of osteoblast activity and differentiation and to prevent stimulation of adipogenesis. As a matter of fact some of these publications recommend genistein dosages and plasma levels that according to the inventors' findings will produce significant adverse results. The inventors have developed a highly reproducible and sensitive in vitro model that has allowed them to discover that the effect of genistein components on e.g. osteogenesis and adipogenesis is biphasic, i.e. at plasma genistein levels above about 0.02 μM estrogen receptors (ER's) may be activated, leading to stimulation of osteogenesis, whilst at plasma genistein levels above about 4 μM the PPARγ receptor is activated, which triggers adipogenesis. By maintaining the plasma genistein levels at a level where the estrogen receptors are continuously activated, whilst at the same time avoiding activation of the PPARγ receptor, disorders such as osteoporosis and obesity may effectively be treated. The inventors have found that at low plasma concentrations (0.02-3 μM) osteogenesis is stimulated and adipogenesis is inhibited by:
1. a stimulation of proliferation of osteoblastic precursor cells
2. a stimulation of differentiation of osteoblasts at the expense of a differentiation in the direction of adipocytes, i.e., a stimulation of osteoblastogenesis and a inhibition of adipogenesis
3. a stimulation of osteoblastic activity.
   These effects are mediated through the ER's.

The investigators have further found that at high genistein concentrations (i.e. above 4 μM, especially above 6 μM), genistein binds to PPARγ, i.e. acts as a true PPARγ agonist. This results in the inhibition of cell proliferation and a decrease in osteoblastogenesis and osteogenesis. At the same time the adipogenesis increases. In addition to its activation of PPARγ activity through direct binding, genistein inhibits MAPK activity, thereby potentiating PPARγ activity. Although at high concentrations ER's are stimulated by genistein, PPARγ activation dominates the biological outcome, i.e. an increase of adipogenesis at the expense of osteoblastogenesis. This results in an “overruling” of the ER mediated effects by PPARγ mediated effects. In addition, there is cross talk between the activity of the receptors, mutually inhibiting each other activities.

It is noted that the aforementioned study by Anderson et al. suggests that high dosages of genistein are less effective than low and intermediate dosages. However, both the intermediate and high dosages employed in said study exceed the dosages encompassed by the present invention.

The aforementioned biphasic dose response of genistein has obvious implications for the use of genistein in the prevention of diseases that are not only estrogen dependent but also dependent on PPARγ activation. In particular in cases where the higher doses lead to undesired inhibition of cellular proliferation and differentiation (i.e, towards adipocytes), care must be taken to ensure that genistein serum levels are maintained within the bandwidth that is defined by the lowest dose that is effective in stimulating an ER, and the lowest dose that is effective in stimulating PPARγ activity. If the doses employed are insufficient to activate an ER (i.e., below 0.02 μM in plasma) there will be no physiological effect. If, however, these doses are too high (i.e., above 4 μM), PPARγ will be activated and the actual effect observed may well be the exact opposite of the effect intended.

Since genistein occurs in varying amounts in a wide array of food products, it is important to realize that blood serum genistein levels are greatly affected by an individual's diet and/or use of nutritional supplements. It has been reported that the plasma concentration of genistein is relatively low and generally less than 20 nM in humans consuming diets without soy. In contrast, it can reach almost 5 μM in the plasma of Japanese who consume high amounts of soy products. Thus, in a method that aims to maintain the blood serum genistein level at a level where it is capable of stimulating osteogenesis and inhibiting adipogenesis, it is crucial to adapt the amount in which genistein is administered to the diet of the individual, to avoid exceeding the upper level above which the opposite effect is observed, due to the activation of PPARγ.

### DETAILED DESCRIPTION OF THE INVENTION

Consequently, the present invention is concerned with a method of controlling the plasma genistein concentration in mammals in order to avoid activation of the peroxisome proliferator-activated receptor γ (PRARγ), said method comprising the steps of:
a. assessing the genistein blood serum concentration of the mammal;
b. if needed, administering to said mammal a genistein component in an amount sufficient to maintain the genistein blood serum concentration at a level between 0.02 and 3 μM, preferably between 0.05 and 2 μM, during at least 8 hours, preferably at least 16 hours of each day;
c. repeating steps a. and b. during a period of at least 30 days with intervals of no more than 3 days.

The term “genistein blood serum concentration” refers to the combined blood serum concentration of pure genistein and certain metabolites of genistein. A suitable method for determining the (combined) genistein blood serum concentration is described by Setchell et al, 2001 J. Nutr. 131:1362-1375S. This method enables the determination of pure genistein as well as of genistein metabolites such as genistein glycons. Throughout this document the terms genistein blood serum concentration and plasma genistein concentration are used interchangeably.

The term “genistein component” encompasses genistein as well as precursors of genistein that are capable of liberating genistein when used in accordance with the present method and derivatives of genistein that display a similar functionality, i.e. that are also capable of activating an ER and PPARγ at similar blood serum concentrations. Whenever reference is made in this document to genistein blood serum concentration it should be understood that this not only relates to the blood serum concentration of genistein per se, but also to the combined blood serum concentration of genistein and of genistein derivatives that display similar in vivo functionality.

The in vivo half-life time of genistein is between 5 and 10 hours and, amongst others, dependent on sex and individual physiology. This is why it is preferred to administer genistein component at least once a day (if needed). If longer administration intervals are employed, relatively high doses are necessary to constantly maintain the genistein blood serum concentration at a sufficiently high level. Such relatively high doses increase the risk that genistein blood serum concentration will exceed 3 μM and thus the risk that the administration of the genistein component will have the opposite effect of what is intended. It is particularly preferred to administer the genistein component at least twice daily.

In a special embodiment of the present invention the method comprises the once daily administration of a slow release formulation comprising the genistein component. The use of a slow release formulation offers the advantage that the genestein blood serum concentration may be maintained at a relatively constant level without the need of administering the genistein component 2, 3 or even more times per day.

It should be understood that if the assessment of the genistein blood serum concentration in step (a) shows said concentration to exceed 3 μM, no genistein will be administered until the day when the estimated concentration is within the range of 0.02 and 3 μM. Thus, the present method also encompasses an administration regimen comprising intervals during which no genistein is administered.

The assessment of the genistein blood serum concentration of a mammal may be done by means of analysis or alternatively by estimating said concentration on the basis of the recent diet of said mammal. Because it is often impractical to carry out regular blood serum analyses to assess the blood serum genistein concentration, it is preferred to assess the genistein blood serum concentration on the basis of the mammal's recent diet. Here by the recent diet is meant the assortment of foodstuffs and nutritional supplements that have been consumed during a period of no more than 4 days prior to the assessment. Preferably the aforementioned assessment is done on an at least once daily basis.

In order to maintain genistein blood serum concentration below 3 μM, the genistein component should generally be administered in an amount which is equivalent to a total daily oral dosage of between 0 and 6 μmole per kg bodyweight, preferably equivalent to a daily oral dosage of between 0 and 2.5 μmole per kg of bodyweight. In most individuals the minimum amount of genistein component that needs to be administered to maintain a sufficiently high genistein blood serum concentration exceeds the equivalent of a daily oral dosage of 0.04 μmole per kg of bodyweight.

In accordance with the present invention the genistein component may be administered in a variety of ways, e.g. enterally or parentally. Most preferably the genistein component is administered orally. The genistein component may suitably be administered orally in the form of a sustained release formulation, which releases the genistein component at such a rate that the genistein blood serum concentration remains within the aforementioned critical ranges. This may be achieved by selecting a sustained release formulation that releases less than 1.3 μmole, more preferably less than 1 mmole genistein component per kg of bodyweight during the first 3 hours after administration.

Preferably the genistein component is administered in the form of a nutritional or pharmaceutical dosage unit that comprises at least 2.8 μmole genistein component, more preferably approximately 10-400 μmole genistein component and most preferably 50-175 μmole genistein component.

Examples of pharmaceutical dosage units that may suitably be used in the present method include tablets, capsules, powders and the like. In an advantageous embodiment of the invention, the pharmaceutical dosage unit is a sustained release formulation. The use of a sustained release formulation offers the advantage that it helps to prevent major fluctuations in genistein blood serum concentrations. Thus the risk that genistein serum concentrations will move outside the desired bandwith is significantly reduced. Examples of suitable sustained release formulations include tablets that comprise a core which contains the genistein component, which core is enveloped by a semi-porous coating (e.g. an acrylate). It is also possible to include the genistein in microcapsules which disintegrate under predefined conditions in the gastrointestinal tract. Furthermore it is feasible to incorporate the genistein component in a pellet which, during passage through the gastointestinal tract disintegrates slowly because of a combination of its size and water solubility. Examples of suitable nutritional dosage units that may suitably be used in accordance with the present invention include cereals, nutritional bars and nutritional drinks.

In accordance with the present method, the genistein component is suitably administered within one hour after the last assessment of the genistein blood serum level. More preferably said administration occurs within half an hour after the assessment.

In a preferred embodiment of the invention step (b) comprises administering the genistein component, if needed, in an amount sufficient to maintain the genistein blood serum concentration at a level between 0.02 and 3 μM. Preferably the genistein component is administered in an amount sufficient to initially raise the genistein blood serum concentration to at least 0.5 μM, preferably at least 0.7 μM, and maintaining said concentration at a level of at least 0.3 μM, more preferably at least 0.4 μM until the next administration.

The present method may suitably be employed to prevent diseases such as osteoporosis and/or obesity by the stimulation of osteogenesis and the inhibition of adipogenesis. The present method is particularly effective in the prevention and treatment of osteoporosis, including postmenopausal osteoporosis, as maintenance of the genistein blood serum concentrations within the advocated bandwidth of 0.02-3 μM, will not only stimulate osteogenesis, but also inhibit adipogenesis. The inhibition of adipogenesis may effectively be used to suppress the formation of body fat in the context of the (prophylactic) treatment of obesity.

The effects of the present invention are most pronounced in tissue in which both estrogen and PPARγ receptors are abundantly present. Examples of such tissues are breast, bone marrow, prostate, vagina, ovaries, uterus and adipose tissue, e.g. in the brain, liver and pancreas.

In order to ensure that the beneficial effects of the present method will last, it is advocated to continue the present method for a period of at least 6 months, more preferably at least 12 months.

A reliable indication as to whether the amount of genistein component that has been administered is sufficiently high can be obtained from assessing the impact on osteogenesis. Preferably, the genistein component, if needed, is administered in an amount sufficient to raise the genistein blood serum concentration to a level where osteogenesis is stimulated. Most preferably osteogenesis is increased by at least 5% relative to the level observed prior to the administration of the genistein component.

It is an essential aspect of the present method that the daily dose of genistein component is adjusted to the intake of genistein through the daily diet. In particular in those cases where the diet leads to substantial fluctuations in the blood serum genistein concentration, the present method can produce particularly good results. Preferably, during the period of at least 30 days, the amount of genistein component that is administered on a daily basis fluctuates between a minimum and a maximum amount that differ by at least a factor 3. More preferably such a differential factor is observed without there being a day during said 30 days when no genistein component was administered.

Even though the present method encompasses regimens with administration free intervals, in a preferred embodiment the genistein component is administered at least once a week. More preferably the genistein component is administered at least 3 days a week. Most preferably the genistein component is administered at least once daily during the period of at least 30 days. Particularly preferred is a method wherein the genistein component is administered at least twice daily. A preferred mode of twice daily administration comprises the administration at a time interval of between 8 and 16 hours. The dosage amounts are preferably adjusted to the time interval between the administrations, meaning that if the next administration is foreseen after e.g. 16 hours, a dose is used which is twice as high as the dose that would be used in case the next administration is foreseen in 8 hours. In a particularly preferred administration regime one dose is administered in the morning (e.g. between 6:00 and 10:00 a.m.) and another dose in the evening (e.g. between 5:00 and 9:00 p.m.). Suitably at least 60 mol % of the daily dosage of the genistein component is administered in the evening.

In a preferred embodiment of the invention the genistein component comprises one or more substances represented by the following formula: 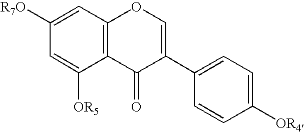
wherein R₅, R₇, and R_{4′} are independently a hydrogen atom; a saturated or unsaturated, linear, branched or cyclic, optionally substituted, alkyl group having from 1 to 6 carbon atoms; an acyl group with a saturated or unsaturated, linear, branched or cyclic, optionally substituted, alkyl radical having from 1 to 8 carbon atoms; a sulphate group; or a mono-, di- or trisaccharide group.

Preferably R₅, R₇, and R_{4′} are independently a hydrogen atom; a saturated, linear or branched alkyl group having from 1 to 3 carbon atoms; an acyl group with a saturated alkyl radical having from 1 to 3 carbon atoms; a sulphate group; or a mono-, di- or trisaccharide group. More preferably R₅, R₇, and R_{4′} are independently a hydrogen atom; a methyl group; an acyl group with a saturated alkyl radical having from 1 to 3 carbon atoms; or a mono- or disaccharide group.

Genistein components wherein at least one of R₅, R₇, and R_{4′} is an alkyl or acyl group, offer the advantage of improved stability and higher activity. Genistein components wherein at least one of R₅, R₇, and R_{4′} is a mono-, di- or trisaccharide group (i.e. glycosylated genistein components) first need to be metabolised into a physiologically active metabolite. Hence such derivatives can offer the advantage of delayed impact.

Glycosylated genistein components that are particularly suited for use in the present method include 7-O-βD-glycoside (genistin). In case the present method employs glycosylated genistein component, it is preferred that at least 50 wt. % of the glycosylated genistein component is a 7-O-βD-glycoside.

Such a delayed impact is particularly advantageous if the genistein component is administered in the evening, particularly between 5:00 and 9:00 p.m. In the morning, however, i.e. between 6:00 and 10:00 a.m., it is advantageous to administer a largely non-glycosylated genistein component as this will result in a more predictable response in terms of blood serum genistein levels. Hence, in a preferred embodiment the present method comprises the steps of administering:
a) at least 0.02 μmole genistein component per kg of bodyweight between 5:00 and 9:00 p.m, wherein at least 70 mol. % of the genistein component is glycosylated; and
b) at least 0.02 μmole genistein component per kg of bodyweight between 6:00 and 10:00 a.m., wherein at most 30 mol. % of the genistein component is glycosylated.

Genistein components may suitably be obtained from natural sources such as soy flour or fermented soy. The application of synthetic genistein and particularly synthetic derivatives of natural genistein is also encompassed by the present invention. Most preferably the present invention employs a genistein component that is obtained from a plant material, preferably the genistein component is obtained from a plant extract, more particularly a proteinaceous plant extract. As mentioned herein before blood serum genistein concentrations are strongly affected by an individual's diet. In particular soy based food products can have a serious impact on said serum concentrations. Consequently, in a preferred embodiment of the present method, step (a) comprises estimating the blood serum genistein concentration on the basis of the amounts of soy based staple food products that were consumed by the mammal during the previous 24 hours. By providing information about the genistein content of such staple food products and taking into account the quantities that were consumed within a given period, it is possible to estimate the genistein blood serum concentration and consequently the amount of genistein component that needs to be administered to maintain optimum genistein blood serum level.

In the present method it may be advantageous to co-administer known active principles against osteoporosis and obesity. Suitable active principles against osteoporosis that may advantageously be administered in combination with the genistein component include polyphenols, vitamin K, zinc, calcium, and/or vitamin D. Preferably the amount of vitamin D which is co-administered does not exceed 1 mg per day, more preferably vitamin D is co-administered in an amount of between 2-10 μg per day. The daily amount of calcium is advantageously kept between 60-1000 mg, preferably between 80-800 calcium. Zinc is suitably administered in a pharmaceutically acceptable form in an amount of no more than 40 mg per day, preferably no more than 30 mg per day. Per dosage (i.e. administration event) the amount of zinc is preferably kept below 15 mg because of the adverse taste effect observed with higher doses. Preferably zinc dosages exceed 5 mg per day. In the prevention of obesity, genistein may be combined with hydroxy citric acid and grapeseed extract.

Another aspect of the invention relates to a method of treating or preventing osteoporosis and/or obesity in mammals, said method comprising the steps of administering:
a) at least 0.02 μmole genistein component per kg of bodyweight between 5:00 and 9:00 p.m, wherein at least 70 mol. % of the genistein component is glycosylated; and
b) at least 0.02 μmole genistein component per kg of bodyweight between 6:00 and 10:00 a.m., wherein at most 30 mol. % of the genistein component is glycosylated.

The advantages of such an administration regimen have been explained herein before. The preferred embodiments of this method are essentially identical to those described in relation to the aforementioned method of controlling the plasma genistein concentration in order to stimulate osteogenesis and to prevent adipogenesis through the activation of PPARγ in mammals.

Another aspect of the invention relates to a pharmaceutical or nutritional kit comprising one or more discrete oral dosage units containing 1.4-200 μmole genistein component, wherein at least 70 mol. % of the genistein component is glycosylated; and one or more discrete oral dosage units containing 1.4-200 μmole genistein component, wherein at most 30 mol. % of the genistein component is glycosylated. Preferably the oral dosage units contain at least 2.8 μmole of the genistein component, more preferably at least 10 μmole. The amount of genistein component in the dosage unit preferably does not exceed 150 μmole. The present kit is preferably provided with instructions for the user indicating that the oral dosage units should be administered in accordance with the method as described herein before. In the present kit, the number of oral dosage units containing at least 70 mol. % of glycosylated genistein and the number of oral dosage units containing not more than 30 mol. % of glycosylated genistein are preferably virtually the same, more preferably, they are exactly the same.

Yet another aspect of the invention relates to an oral dosage unit containing at 1.4-200 μmole genistein component, 2-10 μg vitamin D and 25-3000 μg, preferably 80-800 μg vitamin K. Vitamin D is advantageously employed in the treatment or prevention of osteoporosis as this vitamin is known to enhance calcium absorption. However, vitamin D is also believed to stimulate the formation of osteoclasts. As explained before, the present method aims to promote the formation of osteoblasts whilst at the same time inhibiting the formation of osteoclasts. Thus, the advantageous effect of vitamin D on calcium absorption is offset by its undesirable effect on osteoclast formation. This undesirable effect, however, may be counteracted effectively by incorporating a sufficient amount of vitamin K. Additional advantages associated with the use of vitamin K reside in the vitamins ability to inhibit stimulation of PPARγ and its stimulating effect on osteoblast formation.

Preferably the oral dosage units contain at least 2.8 μmole of the genistein component, more preferably at least 10 μmole. The amount of genistein component in the dosage unit preferably does not exceed 150 μmole. In a preferred embodiment, the oral dosage unit is a discrete solid or semi-solid oral dosage unit such as a tablet or a bar. The amount of genistein component in the oral dosage is preferably within the range of 10-400 μmole.

The invention is further illustrated by means of the following examples.

### EXAMPLES

### Example 1

Effect of Genistein on Osteogenesis

KS 483 cells were continuously treated for 21 days with various concentrations of genistein from day one onwards. Genistein had clear biphasic effects on osteogenesis. The stimulatory effects of genistein on alkaline phosphatase (ALP) activity, nodule formation and Ca deposition were found in the range of 0.1 μM to 10 μM, with a maximal effect at 1 μM. In contrast, the inhibitory effects of genistein on ALP activity, nodule formation and Ca deposition occurred at concentrations of 25 μM or higher. An increase of bone formation at 1 μM and a decrease of bone formation at 25 μM were further confirmed by mRNA expression of the osteoblastic markers, Cbfal, osteocalcin and parathyroid hormone (PTH)/parathyroid hormone related peptide (PTHrP) receptor.

Similar stimulatory and inhibitory effects of genistein on bone formation were also observed in mouse and human bone marrow cell cultures. Like in KS483 cells, the stimulatory effects on ALP activity and Ca deposition in mouse bone marrow cultures occurred at concentrations between 0.1 μM and 10 μM, whereas the inhibitory effects were also found at the concentrations of 25 μM or higher. In human bone marrow cell cultures, the stimulatory effects was found at 0.001 μM, whereas the inhibitory effects were observed above 4 μM.

These findings demonstrate that genistein affects osteogenesis of progenitor cells in a biphasic way, i.e., an increase of osteogenesis at low concentrations and an inhibition of osteogenesis at high concentrations.

Effect of Genistein on_adipogenesis

In contrast to osteogenesis, genistein concurrently inhibited or stimulated adipogenesis in KS483 cells, mouse and human bone marrow cells. Dose-related adipogenic responses in KS483 cells treated with genistein from day 1 onwards showed that genistein decreased adipocyte numbers in the range of 0.1 to 1 μM, whereas it increased adipocyte numbers at concentrations of 10 μM or higher.

These inhibitory and stimulatory effects of genistein on adipogenesis in KS483 cells were further confirmed by mRNA expression of the adipocyte markers, PPARγ2, adipocyte protein 2 (aP2) and lipoprotein lipase (LPL). Adipogenic responses of mouse bone marrow cells to different doses of genistein were obtained. Mouse bone marrow cultures exposed to genistein concentration of 25 μM or higher were not confluent and there were no adipocytes during the cultures. However, an increase in adipocyte numbers was observed at concentrations above 4 μM, whereas a decrease in adipocyte numbers was found at the concentrations of 0.1 and 1 μM.

Similarly, in human bone marrow cultures exposed to different doses of genistein, an increase in adipocyte numbers was observed at the concentration of 10 μM, whereas a decrease of adipocyte numbers was observed at the concentrations of 0.001, 0.01 and 0.1 μM.

Clearly, these data show that genistein affects adipogenesis of progenitor cells in a biphasic way, i.e. an inhibition of adipogenesis at low concentrations and a stimulation of adipogenesis at high concentrations.

Mechanism of Genistein Action on Adipogenesis and Osteogenesis: Effects of Genistein on Transactivation of PPARγ, Binding and MAPK Activity.

KS483 cell were transiently transfected with a luciferase reporter construct containing two copies of a consensus estrogenic receptor responsive element (ERE) inserted in front of TATA or five copies of a consensus PPARy responsive element (PPRE) inserted in front of TATA or empty TATA-luciferase reporter along with expression plasmids encoding human PPARγ2. A dose-dependent increase of ERE reporter activity was found to occur in the genistein concentration range of 0.1 μM to 50 μM. Similarly, a dose-related increase of PPRE reporter activity was found in the range of 1 μM to 50 μM.

The dose-related increase of PPRE reporter activity by genistein was also observed when a luciferase reporter construct containing three copies of a consensus PPRE inserted in front of a minimal thymidine kinase promoter. To determine whether genistein activates PPARγ through direct interaction with this receptor, we performed membrane-bound PPARγ binding assay. Genistein was found to have a measurable Ki of 5.7 μM, which is comparable to the known PPARγ ligand LY 171883 (4.2 μM) and other ligands reported to bind at the micromolar range.

These data demonstrate that genistein can interact directly with the PPARγ ligand binding domain and can thus be defined as a PPARγ ligand. It has been shown that phosphorylation of PPARγ by p42 and p44 mitogen activated protein kinase (MAPK) regulates PPARγ activity. We therefore performed western blot to test whether genistein at the micromolar concentrations inhibit p42 and p44 MAPK activity. An inhibition of p42 and p44 MAPK activity was found to occur in the micromolar range. Our results demonstrate that the transcriptional activation of ERE and PPRE by genistein is dose-dependent but not biphasic. An inhibition of p42 and p44 vMAPK activity is likely to potentiate PPARγ activity.

In addition, we showed here that there is interaction between ER's and PPARγ, possibly responsible for the antiestrogenic effects of genistein. We showed that a co-transfection of PPARγ2 decreased ERE reporter activity when KS483 cells were exposed to the same concentrations of genistein. These results suggest that a competition of the common ligand genistein might occur between ER's and PPARγ. It is also possible that an activation of PPARγ down regulated transcriptional activity of estrogen, i.e. there is cross-talk between ER's and PPARγ. Co-transfection of ERα in KS483 cells exposed to genistein resulted in a down regulation of PPRE reporter activity. These results indicate that either a competition for the same ligand occurred between ER's and PPARγ or a suppressive function of ERα on PPARγ transcriptional activity. Different from the biphasic effects of genistein on osteogenesis and adipogenesis of KS483 cells, both ERE and PPRE reporter activities showed a dose-related increase and reach the maximal level at genistein concentration of 50 μM. The difference between biological effects and the transcriptional activation indicates that biological effects of genistein depend on the balance of an activation between ER-dependent pathway and PPARγ-dependent pathway. Ligand concentration thus plays a crucial role in determining the degree of activation of ER's and PPARγ.

In conclusion, our results demonstrate that the transcriptional activation of ERE and PPRE by genistein is dose-dependent but not biphasic and that there are cross talks between ER's and PPARγ.

### Example 2

Two different types of tablets are prepared for use in a method of treating osteoporosis and/or obesity. One tablet is designed for administration in the morning between 6:00 and 10:00 a.m. (“morning tablet”), the other is other tablet is intended for administration in the evening between 5:00 and 9:00 p.m. (“evening tablet”).

The formulations of these 2 tablets are as follows:

Morning Tablet (1 gram):

| | |
|---|---|
| Genistein | 14 mg |
| Vitamin K | 0.1 mg |
| Vitamin D | 5 μg |
| Calcium carbonate | 500 mg |
| Excipient | remainder |

Evening Tablet (2 gram):

| | |
|---|---|
| Soy extract | 500 mg (36 mg, mainly |
| | glycosylated, genistein) |
| Vitamin K | 0.1 mg |
| Vitamin D | 5 μg |
| Calcium carbonate | 400 mg |
| Zinc sulphate monohydrate | 66 mg |
| Excipient | remainder |

### Example 3

A sustained release tablet is prepared for use in a method of treating osteoporosis and/or obesity from the following ingredients:

Tablet core

| | |
|---|---|
| Genistein | 30 mg |
| Metolose 60SH4000 | 189.1 mg |
| Magnesium stearate | 0.9 mg |
| Subtotal | 220.0 mg |

Coating

| | |
|---|---|
| ETHOCEL 10 (Dow Chemcial) | 5.0 mg |
| Polyethylene glycol 6000 | 0.5 mg |
| Subtotal | 5.5 mg |
| Total | 225.5 mg |

To the genistein are added Metolose 60SH4000 and magnesium stearate, and the mixture is thoroughly mixed. The mixture is directly compress-formed by a continuous compressor equipped with a 8 mm diameter, 6.5 R pounder under the main pressure of 1 ton into tablets each weighing 220 mg. Then, the obtained base tablets are placed in a pan coater (Freund Industry) and a solution of ETHOCEL 10 and polyethylene glycol 6000 in a mixture of water-ethanol (1:9) is sprayed thereon, followed by drying to give the filmn coated tablets.

1-15. (canceled)

16. A method for the treatment or prevention of osteoporosis and/or obesity, while preventing a decrease in osteogenesis and an increase in adipogenesis, said method comprising the steps of:
a) assessing the genistein blood serum concentration of a mammal;
b) if the blood serum level of a) is below 3 μM, administering to said mammal a genistein component in an amount sufficient to maintain the genistein blood serum concentration at a level between 0.02 and 3 μM during at least 8 hours, preferably at least 16 hours of each day; if the blood serum level of a) exceeds 3 μM, not administering to said mammal a genistein component until the blood serum level is below 3 μM;
c) repeating steps a. and b. during a period of at least 30 days with intervals of no more than 3 days.

17. A method according to claim 16, whereby the genistein blood serum concentration in step a. is assessed on the basis of the recent diet of said mammal.

18. A method according to claim 16, whereby the genistein component in step b. is administered to the mammal in an amount sufficient to raise the genistein blood serum concentration to a level where osteogenesis is stimulated.

19. A method according to claim 16, whereby the genistein component is administered in step b. in an amount which is below the amount that would increase the genistein blood serum concentration to a level where activation of PPARγ causes adipogenesis.

20. A method according to claim 16, whereby during the period of at least 30 days the amount of genistein component that is administered on a daily basis fluctuates between a minimum and a maximum amount that differ by at least a factor 3.

21. A method according to claim 16, whereby the genistein component is administered at least once a week.

22. A method according to claim 16, whereby the genistein component comprises one or more substances represented by the following formula: 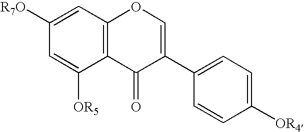
wherein R₅, R₇, and R_{4′} are independently a hydrogen atom; a saturated or unsaturated, linear, branched or cyclic, optionally substituted, alkyl group having from 1 to 6 carbon atoms; an acyl group with a saturated or unsaturated, linear, branched or cyclic, optionally substituted, alkyl radical having from 1 to 8 carbon atoms; a sulphate group; or a mono-, di-or trisaccharide group.

23. A method according to claim 16, whereby the genistein component is administered orally.

24. A method according to claim 16, whereby step a. comprises estimating the blood serum genistein concentration on the basis of the amounts of soy based staple food products that were consumed by the individual during the previous 24 hours.

25. A method according to claim 16, whereby the genistein component is administered in an amount equivalent to a daily oral dosage which is within the range of 0 to 3 μmoles per kg bodyweight.

26. A method of for treating or preventing osteoporosis and/or obesity in mammals, said method comprising the steps of administering:
a) at least 0.02 mmole genistein component per kg of bodyweight between 5:00 and 9:00 p.m, whereby at least 70 mol. % of the genistein component is glycosylated; and
b) at least 0.02 μmole genistein component per kg of bodyweight between 6:00 and 10:00 a.m., whereby at most 30 mol. % of the genistein component is glycosylated.

27. A method according to claim 26, whereby at least 60 mol % of the daily dosage of the genistein component is administered in the evening.

28. A pharmaceutical or nutritional kit comprising one or more discrete oral dosage units containing 1.4-200 μmole genistein component, whereby at least 70 mol. % of the genistein component is glycosylated; and one or more discrete oral dosage units containing at 1.4-200 mmole genistein component, whereby at most 30 mol. % of the genistein component is glycosylated.

29. An oral dosage unit containing 1.4-200 μmole genistein component, 2-10 μg vitamin D and 25-3000 μg vitamin K.

30. A method according to claim 16, whereby the genistein component is in the form of a sustained release formulation, which releases less than 1.3 μmole genistein component per kg body weight during the first three hours after administration.

31. An oral dosage unit comprising a genistein component in the form of a sustained release formulation, which releases less than 1.3 μmole genistein component per kg body weight during the first three hours after administration and which comprises the genistein component in an amount sufficient to maintain the genistein blood serum concentration at a level between 0.02 and 3 μM during at least 8 hours, preferably at least 16 hours of each day.

32. The oral dosage unit according to claim 31, whereby said dosage unit further comprises 80-800 mg calcium and 5-15 mg zinc.
